(19) United States
(12) Patent Application Publication
(10) Pub. No.: US 2026/0256942 A1
(43) Pub. Date: Sep. 3, 2026

(54) Nanocarrier Composition with Adjustable Structure, Preparation Method and Use Thereof

(71) Applicant: Powin Biomedical Co., Ltd., Taichung City (TW)

(72) Inventors: Syu Ming Lai, Taichung City (TW); Cun-Zhao Li, Dacun Township (TW); Po Cheng Chu, Taoyuan City (TW); Man Hua Liao, Taichung City (TW); Ping Shan Lai, Taichung City (TW)

(21) Appl. No.: 19/539,895

(22) Filed: Feb. 13, 2026

(30) Foreign Application Priority Data
Feb. 18, 2025 (TW) 114105859

(51) Int. Cl. A61K 47/69; A61K 9/127; A61K 9/51; A61K 31/00

(52) U.S. Cl. CPC A61K 47/6907; A61K 9/127; A61K 9/5153; A61K 31/00

(57) ABSTRACT

The present disclosure discloses a nanocarrier composition with adjustable structure, preparation method and use thereof. The nanocarrier composition is prepared by adding a regulator to lecithin. By adding different types of regulators, the nanocarrier composition can be converted between a liposome structure or a micelle structure. When applied to medical purposes, under the same nanocarrier composition preparation process, under the same nanocarrier composition manufacturing process, the structure of the nanocarrier composition can be changed by simply changing the type of added regulator, thereby controlling the drug release time, drug penetration and bioavailability. The nanocarrier composition can be converted into a suitable structure to facilitate drug delivery according to the requirements of different dosage forms.

### CROSS-REFERENCE TO RELATED APPLICATION

This application claims the benefit of Taiwan Patent Application No. 114105859, filed on Feb. 18, 2025, in the Taiwan Intellectual Property Office, the entire disclosure of which is hereby incorporated by reference in its entirety.

### BACKGROUND OF THE INVENTION

### 1. Field of the Invention

The present disclosure discloses a nanocarrier composition with adjustable structure, a preparation method and use thereof, and particularly relates to a nanocarrier composition with adjustable structure for drug delivery, a preparation method and use thereof.

### 2. Description of the Related Art

Lecithin is a natural phospholipid, mainly derived from soybeans or egg yolks. It plays a key role in the preparation of liposomes since the phospholipid bilayer structure of lecithin enables it to self-assemble into lipid vesicles.

Liposomes are spherical vesicles surrounded by a phospholipid bilayer membrane which are usually composed of phospholipid molecules; these molecules spontaneously form a closed bilayer structure in an aqueous solution. The formation mechanism is that when phospholipid molecules are dispersed in an aqueous solution, due to their amphiphilic characteristics (hydrophilic head and hydrophobic tail), they will spontaneously organize into a phospholipid bilayer membrane structure, which may be used as a carrier for encapsulating water-soluble and fat-soluble molecules, and therefore has potential applications in drug delivery.

Micelles, on the other hand, are smaller spherical structures formed by surfactant molecules, which typically consist of a single layer of molecules with the hydrophobic tails facing inward and the hydrophilic heads facing outward. The formation mechanism is that when the surfactant concentration exceeds the critical micelle concentration (CMC), the molecules spontaneously aggregate into a micelle structure, with the hydrophobic portion in contact with oil, while the hydrophilic portion interacts with water. Therefore, the core of a micelle is typically hydrophobic, while the exterior is hydrophilic. This structure enables micelles to encapsulate hydrophobic molecules, making them commonly used as carriers for lipid-soluble drugs.

In general, micelles are relatively easy to prepare, but their structure is relatively unstable. When stored for long periods of time or under certain environmental conditions (such as high temperature or low pH), micelles may decompose or aggregate. In contrast, liposomes generally have good stability. Since the structure of the phospholipid molecules provides enhanced protection, they can preserve drugs or active ingredients for a long time. This makes liposomes more advantageous in terms of preparation and long-term storage.

The applications of liposomes and micelles in drug delivery systems have been extensively studied, and each has unique characteristics and differences in terms of structure, properties, and applications. Micelles are commonly used in cosmetics, drug delivery systems, and biochemical research, where rapid release of drugs or active ingredients is required. Liposomes, on the other hand, are widely used in drug delivery, biomedical research, and other fields, particularly those requiring stable, long-term delivery. Therefore, liposomes are suitable for controlled drug release, increasing drug bioavailability, and improving drug stability.

Therefore, micelles and liposomes are different nanoscale carrier systems, each with its own characteristics in structure, drug delivery, stability and application areas. The question of which system to choose depends on the specific application requirements and the required efficacy.

In summary, liposomes and micelles differ in drug loading capacity, release efficiency, and tissue distribution. Therefore, a carrier microstructure that can be easily regulated by a regulator is needed, which can then be differentiated according to the loading requirements of different drugs, thereby achieving the requirement of better drug delivery efficiency.

### SUMMARY OF THE INVENTION

In view of the above-mentioned shortcomings of the prior art, the inventors of the present application have been eager to improve and innovate, and have developed a nanocarrier composition with adjustable structure. The nanocarrier composition is formed by adding a regulator to lecithin. By controlling the type and ratio of the regulator, the nanocarrier composition can be converted between liposomes and micelles, thereby changing its nanostructural properties to suit the encapsulation requirements of different substances. When used as a nanocarrier for drugs, it provides better drug delivery efficiency when administered orally or transdermally.

The present disclosure regulates the microstructure of the nanocarrier by adding a regulator, enabling precise control of drug release rate, permeability, and bioavailability. This is of great significance for improving therapeutic efficacy and reducing side effects. For example, when liposomes are used as drug carriers, they exhibit sustained-release properties and reduced toxicity, which is suitable for systemic drug delivery systems for antifungal drugs (such as amphotericin B) and anticancer drugs (such as daunorubicin or doxorubicin). When micelles are used as drug carriers, they provide faster drug release and enhanced drug permeability through the intestinal lining. Therefore, micelles are commonly used in oral drug delivery systems for drugs such as BCS class II drugs (such as metoprolol) and BCS class IV drugs (such as hydrochlorothiazide).

The regulator used in the present disclosure is a terpene. Terpenes are a class of hydrocarbons widely found in nature. Terpenes are hydrocarbons that can be divided into several isoprene structural units. Their molecular formula are simple multiples of the isoprene unit, and the general formula can be written as (C₅H₈)ₙ. Initially, it was believed that terpenes were derived from isoprene (C₅H₈), primarily produced by plants, and are responsible for the unique aroma of plants such as pine trees, flowers, and spices. Terpenes can be divided into multiple categories based on their structure, including monoterpenes, sesquiterpenes, and diterpenes. These compounds are widely used in industries such as perfumes, food additives, medicine, and aromatherapy.

Among them, terpenes may be used to adjust the assembly behavior and physicochemical properties of liposomes such as lecithin due to their hydrophobic characteristics and ability to interact with other molecules; for example, terpenes can be embedded within the bilayer membrane formed by lecithin, thereby modulating membrane fluidity and permeability. This modulation increases the bilayer thickness and enhances the structural regularity of the membrane, in a manner that directly correlates with enhanced encapsulation efficiency (EE), improved drug loading capacity (DL), and prolonged structural stability under storage conditions, as evidenced by transmission electron microscopy observations. These structural changes, in turn, improve coating characteristics and release kinetics, facilitate enhanced drug permeation across the liposomal membrane, and contribute to higher encapsulation efficiency, greater structural stability, and improved bioavailability. Such modulation facilitates drug permeation through the liposomal membrane, enhances drug loading efficiency, and improves the overall structural stability of the liposomal carrier.

Furthermore, terpene compounds with specific structures can induce structural transformation of liposomes, transitioning from a bilayer vesicular configuration to a micellar configuration. Furthermore, due to the hydrophobic properties of terpene compounds, they can interact with the hydrophobic tail of lecithin, thereby altering the size, shape and surface properties of micelles.

On the other hand, since liposomes have sustained-release properties and the ability to reduce toxicity when used as drug carriers, they are more suitable for use as carriers for oral drugs. While loading the drug, they form a physical barrier to protect the drug from being destroyed by gastric acid and digestive enzymes, so that the drug's efficacy is not affected by the gastrointestinal environment. They can also promote the absorption of drugs through intestinal epithelial cells, increase the effective concentration of drugs in the body, and improve the bioavailability of drugs.

Furthermore, since micelles can interact with the lipids in the stratum corneum of the skin, temporarily disrupting the structure of the stratum corneum to reduce the barrier function of the skin, thereby enhancing the ability of drugs to penetrate the skin, therefore more suitable as carriers for topical medications and can provide faster drug release. Furthermore, the hydrophobic core of micelles can encapsulate fat-soluble drugs, increasing their solubility in the formulation and thereby improving transdermal absorption.

A first object of the present disclosure is to provide a nanocarrier composition with adjustable structure. The nanocarrier composition comprises lecithin, a regulator, a surfactant, and sufficient amount of water. Based on the total weight of the nanocarrier composition, the content of lecithin is 1-5 wt %, the content of the regulator is 0.5-5 wt %, the content of the surfactant is 1-5 wt %, and the content of water is 85-97.5 wt %.

Preferably, based on the total weight of the nanocarrier composition, the content of lecithin is 1-3 wt %, the content of the regulator is 1 wt %, the content of the surfactant is 1-3 wt %, and the content of water is 93-97 wt %.

The regulator is a terpene compound, and the terpene compound is selected from a monocyclic terpene compound, a bicyclic terpene compound or a chain terpene compound. The monocyclic terpene compound is selected from D-limonene, phellandrene, pulegone, sabinene, terpinene, DL-menthol, terpineol, humulene, (−)-α-bisabolol, but not limited thereto; the bicyclic terpene compound is selected from camphene, pinene, fenchone, (−)-borneol, camphor, fenchol, trans-caryophyllene, valencene, caryophyllene oxide, guaiac, cedrene, (+)-cedrol, but not limited thereto; and the chain terpene compound is selected from linalool, nerol, β-myrcene, ocimene, nerolidol, farnesene, but not limited thereto.

Preferably, the monocyclic terpene compound is selected from (−)-α-bisabolol, DL-menthol, and D-limonene; the bicyclic terpene compound is selected from (+)-cedrol, trans-caryophyllene, and (−)-borneol; and the chain terpene compound is selected from β-myrcene, farnesene, ocimene, and linalool.

Preferably, the terpene compound is a monocyclic terpene compound or a bicyclic terpene compound when the nanocarrier composition is to form a liposome structure; more preferably, the terpene compound is D-limonene.

More preferably, the terpene compound is a chain terpene compound when the nanocarrier composition is to form a micelle structure; more preferably, the terpene compound is linalool.

The surfactant is any one of anionic surfactants, cationic surfactants, zwitterionic surfactants, nonionic surfactants or ionic surfactants, or a combination thereof; preferably, the surfactant is selected from any one of polysorbate, polyethylene glycol, polymethyl methacrylate, polyglycolide, polylactide, polycaprolactone, polylactide-polyethylene glycol, polycaprolactone-polyethylene glycol, vitamin E polyethylene glycol succinate, poloxamer or polyoxyethylene hydrogenated castor oil, or a combination thereof; more preferably, the surfactant is polysorbate, such as polysorbate 20 (Tween 20) or polysorbate 80 (Tween 80); further preferably, the surfactant is polysorbate 80.

The nanocarrier composition has an effective average particle size of less than about 1,000 nm; preferably, the effective average particle size is about 30-700 nm; more preferably, the effective average particle size is about 50-450 nm; further preferably, the effective average particle size is about 50-400 nm.

The effective polymer dispersion index (PDI) of the nanocarrier composition is about 0-1; preferably, the effective polymer dispersion index is about 0-0.6; more preferably, the effective polymer dispersion index is about 0.05-0.5.

The nanocarrier composition has an effective zeta potential range of about 0 to −100 mV; preferably, the effective zeta potential range is about 0 to −50 mV; and more preferably, the effective zeta potential range is about −10 to −50 mV. Within this specific zeta potential range, the nanocarrier composition maintains a stable structural morphology and can remain unchanged for several months, preferably for about three months.

The effective encapsulation efficiency (EE) of the nanocarrier composition is in the range of about 30-100%. This range ensures sufficient drug entrapment to achieve therapeutically relevant dosages while maintaining particle stability during long-term storage. Preferably, the effective encapsulation efficiency is in the range of about 50-100%. More preferably, the effective encapsulation efficiency is in the range of about 70-100%.

The effective drug loading (DL) of the nanocarrier composition is in the range of about 0.1-50%; preferably, the effective drug loading is in the range of about 5-40%; more preferably, the effective drug loading is in the range of about 20-30%.

A second object of the present disclosure is to provide a method for preparing a nanocarrier composition containing terpene compounds, comprising the following steps:
Step S1: dissolving lecithin, a terpene compound and a surfactant in an organic solvent to form a mixed solution;
Step S2: evaporating and removing the organic solvent in the mixed solution to form a thin film containing lecithin, the regulator, and the surfactant; and
Step S3: adding water to dissolve the film back, and applying energy to perform a hydration reaction to obtain a nanocarrier composition containing the regulator.

A third object of the present disclosure is to provide a nanocarrier composition with adjustable structure for use as a nanocarrier for loading drugs, wherein the drug is selected from the group consisting of cannabinoids, flavonoids, carotenoids, triterpenoids, polyphenols, alkaloids, fatty acids, fat-soluble vitamins, polysaccharides, volatile oils, and amino acid compounds.

When the loaded drug is to be administered orally, the terpene compound contained in the nanocarrier composition is a monocyclic terpene compound or a bicyclic terpene compound, and the nanocarrier composition exhibits a liposome structure. When the loaded drug is to be administered transdermally, the terpene compound contained in the nanocarrier composition is a chain terpene compound, and the nanocarrier composition exhibits a micelle structure.

The present disclosure demonstrates for the first time that the addition of terpenes significantly enhances the outer bilayer structure of liposomes, resulting in increased bilayer thickness, improved structural regularity, and enhanced stability. These improvements directly contribute to higher encapsulation efficiency (EE) and drug loading capacity (DL), as well as extended storage stability of the nanocarrier. This enhancement not only increases the thickness and stability of the outer ring, but also effectively improves drug encapsulation efficiency and the long-term stability of the carrier. In summary, the nanocarrier composition with adjustable structure provided by the present disclosure can be regulated by the type of terpenes contained to form liposomes or micelles. Depending on the characteristics of the liposomes or micelles, the encapsulated drug can be administered orally or transdermally, thereby improving drug delivery efficiency and bioavailability.

The following is a detailed description of specific embodiments with reference to the accompanying drawings for better understanding of the purpose, technical content, features and effects achieved by the present disclosure.

### BRIEF DESCRIPTION OF THE DRAWINGS

FIG. 1A is a schematic diagram of the structure of a nanocarrier composition containing (−)-α-bisabolol, observed under a transmission electron microscope without negative staining.

FIG. 1B shows a schematic diagram of the structure of a nanocarrier composition containing farnesene, observed under a transmission electron microscope without negative staining.

FIG. 1C shows a schematic diagram of the structure of a nanocarrier composition containing (−)-borneol, observed under a transmission electron microscope without negative staining.

FIG. 1D shows a schematic diagram of the structure of a lecithin liposome without terpene compounds, observed under a transmission electron microscope without negative staining.

FIG. 2A is a schematic diagram of the structure of lecithin liposomes without terpene compounds, observed under an electron microscope under negative staining.

FIG. 2B shows a schematic diagram of the structure of the nanocarrier composition containing D-limonene, observed under an electron microscope under negative staining.

FIG. 2C shows a schematic diagram of the structure of the nanocarrier composition containing linalool, observed under an electron microscope under negative staining.

FIG. 3A is a graph showing the analysis of the change in particle size of the nanocarrier composition containing a cyclic terpene compound and a linear terpene compound of the present disclosure over a period of three months.

FIG. 3B is a graph showing the analysis of the change in polymer dispersion index of the nanocarrier composition containing a cyclic terpene compound and a linear terpene compound of the present disclosure over a period of three months.

### DETAILED DESCRIPTION OF THE PREFERRED EMBODIMENTS

To clearly illustrate the features, aspects, advantages, and effects achieved by the present disclosure, the present disclosure is described in detail with reference to the accompanying drawings. The figures used herein are for illustrative purposes only and to assist in the description, and may not reflect the actual scale and precise configuration of the present disclosure after implementation. Therefore, it should be noted that the scale and configuration of the accompanying drawings should not be used to limit the scope of the present disclosure in actual implementation.

For a more complete and clear understanding of the technical content, inventive objectives and effects achieved by the present disclosure, embodiments are described in detail below with reference to the disclosed drawings.

### Preparation Example 1

The nanocarrier composition with adjustable structure is prepared through the following steps:

Step S1: dissolving lecithin, a regulator and a surfactant in an organic solvent to form a mixed solution.

In step S1, the weight ratio of lecithin, the regulator, and the surfactant is 1-3:1:1-3, and the amount of the organic solvent is not particularly limited as long as the lecithin, the regulator, and the surfactant can be completely dissolved.

The regulator is a terpene compound, and the terpene compound is selected from a monocyclic terpene compound, a bicyclic terpene compound or a chain terpene compound;

wherein the monocyclic terpene compound is selected from (−)-α-bisabolol, DL-menthol, and D-limonene, the bicyclic terpene compound is selected from (+)-cedrol, trans-caryophyllene, and (−)-borneol, and the chain terpene compound is selected from β-myrcene, farnesene, ocimene, and linalool; the monocyclic, bicyclic or chain terpene compound is not limited to the specific terpene compounds mentioned above, and all types of monocyclic, bicyclic or chain terpene compounds may be used.

Preferably, the terpene compound is a monocyclic terpene compound or a bicyclic terpene compound when the nanocarrier composition is to form a liposome structure; more preferably, the terpene compound is D-limonene.

More preferably, the terpene compound is a chain terpene compound when the nanocarrier composition is to form a micelle structure; more preferably, the terpene compound is linalool.

The organic solvent is selected from any one of methanol, n-hexane, ethanol, isopropanol, acetone, formic acid, acetic acid, acetonitrile, dichloromethane, dimethylformamide or tetrahydrofuran, or a combination thereof; preferably, the organic solvent is methanol.

The surfactant is any one of anionic surfactants, cationic surfactants, zwitterionic surfactants, nonionic surfactants or ionic surfactants, or a combination thereof; preferably, the surfactant is selected from any one of polysorbate, polyethylene glycol, polymethyl methacrylate, polyglycolide, polylactide, polycaprolactone, polylactide-polyethylene glycol, polycaprolactone-polyethylene glycol, vitamin E polyethylene glycol succinate, poloxamer or polyoxyethylene hydrogenated castor oil, or a combination thereof; more preferably, the surfactant is polysorbate, such as polysorbate 20 (Tween 20) or polysorbate 80 (Tween 80); further preferably, the surfactant is polysorbate 80.

Step S2: evaporating and removing the organic solvent in the mixed solution to form a thin film containing lecithin, a regulator and a surfactant.

In step S2, the temperature for evaporating the organic solvent is 40-70° C.; preferably, the temperature for evaporating the organic solvent is 55° C.

Step S3: adding water to dissolve the film back, and applying energy to perform a hydration reaction to obtain a nanocarrier composition containing a regulator.

In step S3, the weight ratio of the added water to the total weight of the lecithin, the regulator, and the surfactant is 93:7-97:3, and the time for applying energy to perform the hydration reaction is 5-15 minutes.

The energy application method includes stirring, vortex oscillation or ultrasonication in a water bath; preferably, the energy application method is ultrasonication in a water bath.

In addition, similar to the above preparation steps, lecithin liposomes were prepared using only lecithin and surfactant without adding the regulator (ie, terpene compound) to serve as a control group for subsequent nanocarrier compositions containing the regulator.

### Example 1

The specific type of the regulator used in the nanocarrier composition containing the regulator obtained by the method described in Preparation Example 1 (i.e., the type of terpene compound), as well as the ratios of lecithin, regulator, and surfactant, are shown in Table 1. Furthermore, the particle size and polymer dispersion index (PDI) of these nanocarrier compositions containing regulators were measured using dynamic light scattering (DLS), and the results are also shown in Table 1.

TABLE 1

| | | | | | | Particle | |
|---|---|---|---|---|---|---|---|
| | | Terpenes | Lecithin | Tween 80 | water | size | |
| Group | | (wt %) | (wt %) | (wt %) | (wt %) | (nm) | PDI |
| 1 | (−)-α- | 1 | 1 | 1 | 97 | 111 | 0.259 |
| | Bisabolol | 1 | 2 | 2 | 95 | 286.2 | 0.474 |
| | | 1 | 3 | 3 | 93 | 222.2 | 0.574 |
| 2 | β-Myrcene | 1 | 1 | 1 | 97 | 139.7 | 0.447 |
| | | 1 | 2 | 2 | 95 | 101.5 | 0.368 |
| | | 1 | 3 | 3 | 93 | 121.9 | 0.488 |
| 3 | DL-Menthol | 1 | 1 | 1 | 97 | 117 | 0.411 |
| | | 1 | 2 | 2 | 95 | 162.6 | 0.545 |
| | | 1 | 3 | 3 | 93 | 134.4 | 0.538 |
| 4 | (+)-Cedrol | 1 | 1 | 1 | 97 | 438.9 | 1 |
| | | 1 | 2 | 2 | 95 | 384 | 0.625 |
| | | 1 | 3 | 3 | 93 | 2129 | 1 |
| 5 | Farnesene | 1 | 1 | 1 | 97 | 152.8 | 0.266 |
| | | 1 | 2 | 2 | 95 | 202.2 | 0.451 |
| | | 1 | 3 | 3 | 93 | 153.8 | 0.496 |
| 6 | Ocimene | 1 | 1 | 1 | 97 | 140.2 | 0.425 |
| | | 1 | 2 | 2 | 95 | 135.5 | 0.479 |
| | | 1 | 3 | 3 | 93 | 145.1 | 0.509 |
| 7 | trans- | 1 | 1 | 1 | 97 | 220 | 0.489 |
| | caryophyllene | 1 | 2 | 2 | 95 | 148.3 | 0.449 |
| | | 1 | 3 | 3 | 93 | 240.4 | 0.525 |
| 8 | (−)-Borneol | 1 | 1 | 1 | 97 | 114 | 0.408 |
| | | 1 | 2 | 2 | 95 | 154.1 | 0.32 |
| | | 1 | 3 | 3 | 93 | 203.7 | 0.639 |
| 9 | D-limonene | 1 | 1 | 1 | 97 | 71.31 | 0.248 |
| | | 1 | 2 | 2 | 95 | 115.6 | 0.431 |
| | | 1 | 3 | 3 | 93 | 73.74 | 0.437 |
| 10 | Linalool | 1 | 1 | 1 | 97 | 143.3 | 0.308 |
| | | 1 | 2 | 2 | 95 | 89.83 | 0.384 |
| | | 1 | 3 | 3 | 93 | 98.33 | 0.428 |
| control | — | 0 | 1 | 1 | 98 | 109.1 | 0.295 |
| group | | 0 | 2 | 2 | 96 | 71.62 | 0.589 |
| | | 0 | 3 | 3 | 94 | 126.5 | 0.504 |

The results in Table 1 show that different terpenes, lecithin, and surfactant ratios all significantly affect the size and uniformity of the nanocarriers. A smaller PDI value indicates a more uniform molecular weight distribution of the polymer within the nanocarriers.

Next, representative groups were selected for transmission electron microscopy (TEM) observation to gain a deeper understanding of the microscopic morphology and structural characteristics of the nanocarriers.

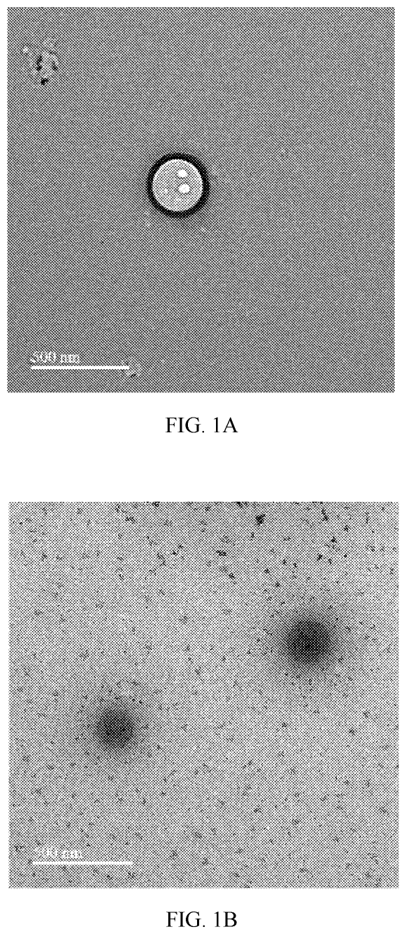

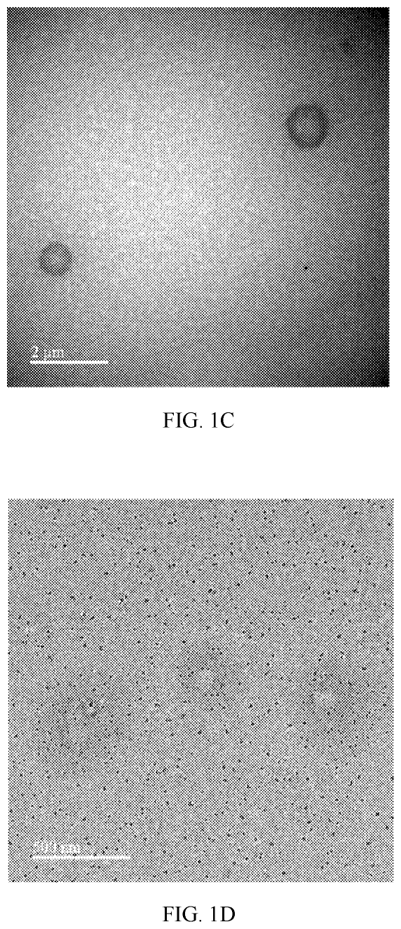

Please refer to FIG. 1A to FIG. 1D, which show the structures of the nanocarrier compositions of the present disclosure containing monocyclic terpene compounds, linear terpene compounds, and bicyclic terpene compounds, as well as lecithin liposomes without terpene compounds, as observed under a transmission electron microscope without adding a negative stain. FIG. 1A shows a nanocarrier composition containing (−)-α-bisabolol, FIG. 1B shows a nanocarrier composition containing farnesene, FIG. 1C shows a nanocarrier composition containing (−)-borneol, and FIG. 1D shows a lecithin liposome without terpene compounds.

As can be seen in FIG. 1A to FIG. 1D, nanocarrier compositions containing terpene compounds of varying structures exhibit different microstructures. Since liposomes are primarily spherical vesicles surrounded by a high-electron-density phospholipid bilayer, a membrane structure can be clearly seen on the periphery (as shown in FIGS. 1A, 1C, and 1D). Micelles, on the other hand, are primarily aggregated spherical structures lacking an outer membrane structure, resulting in the overall appearance of a dark grayscale sphere (as shown in FIG. 1B).

Therefore, the above results show that nanocarrier compositions containing monocyclic or bicyclic terpene compounds exhibit a liposome structure, similar to lecithin liposomes without terpene compounds, while nanocarrier compositions containing linear terpene compounds exhibit a micellar structure. This confirms that the structure of terpene compounds has a significant impact on the form of the nanocarriers. By adding cyclic or linear terpene compounds to lecithin, the microstructure of the nanocarrier composition can be manipulated, thereby enabling carrier design for different drug delivery requirements.

### Example 2

Based on the experimental results obtained in Example 1, terpene compounds with similar chemical structures were further selected for comparison. The terpene compounds used for comparison here were cyclic D-limonene and chain-structured linalool. Both were then loaded with the active ingredient cannabidiol (CBD) to compare their encapsulation efficiency (EE) and drug loading (DL).

Please refer to Table 2, which shows the experimental results of particle size, polymer dispersion index (PDI), and zeta potential when cyclic D-limonene is added to lecithin to form a liposome-structured nanocarrier composition, at different usage ratios of the various components.

TABLE 2

| | | | | | | | Zeta |
|---|---|---|---|---|---|---|---|
| | Lecithin | Terpenes | Tween 80 | Water | Particle size | | potential |
| Group | (wt %) | (wt %) | (wt %) | (wt %) | (nm) | PDI | (mV) |
| 1-1 | 1 | 1 | 1 | 97 | 71.31 ± 0.827 | 0.248 ± 0.007 | −29.1 ± 0.208 |
| 1-2 | 1 | 3 | 1 | 95 | 121.4 ± 3.121 | 0.264 ± 0.007 | −44.5 ± 1.40 |
| 1-3 | 1 | 4 | 1 | 94 | 398.2 ± 45.03 | 0.441 ± 0.037 | −21.9 ± 1.02 |
| 1-4 | 1 | 1 | 3 | 95 | 117.7 ± 1.626 | 0.292 ± 0.003 | −17.8 ± 0.75 |
| 1-5 | 1 | 1 | 4 | 94 | 132.8 ± 0.208 | 0.641 ± 0.008 | −18.4 ± 0.115 |
| 1-6 | 2 | 1 | 1 | 96 | 265.8 ± 9.755 | 0.661 ± 0.079 | −32.5 ± 0.458 |
| 1-7 | 3 | 1 | 1 | 95 | 347.6 ± 7.504 | 0.952 ± 0.080 | −27.3 ± 1.08 |
| 1-8 | 1 | 3 | 3 | 93 | 73.74 ± 0.743 | 0.437 ± 0.004 | −42.5 ± 0.306 |

From Table 2, when using cyclic D-limonene, considering the comprehensive results of particle size, polymer dispersion index, and zeta potential, the nanocarrier composition of Group 1-8 was selected for subsequent experiments.

Please refer to Table 3, which shows the experimental results of particle size, polymer dispersion index (PDI), and zeta potential when chain-structured linalool is added to lecithin to form a liposome-structured nanocarrier composition, at different usage ratios of the various components.

TABLE 3

| | | | | | | | Zeta |
|---|---|---|---|---|---|---|---|
| | Lecithin | Terpenes | Tween 80 | water | Particle size | | potential |
| Group | (wt %) | (wt %) | (wt %) | (wt %) | (nm) | PDI | (mV) |
| 2-1 | 1 | 1 | 1 | 97 | 143.3 ± 2.003 | 0.308 ± 0.006 | −32.7 ± 0.057 |
| 2-2 | 1 | 2 | 1 | 96 | 247.5 ± 1.904 | 0.362 ± 0.049 | −22.6 ± 0.451 |
| 2-3 | 1 | 3 | 1 | 95 | 644.1 ± 6.035 | 0.546 ± 0.011 | −9.67 ± 0.411 |
| 2-4 | 1 | 1 | 2 | 96 | 95.5 ± 1.915 | 0.374 ± 0.017 | −16.5 ± 0.458 |
| 2-5 | 1 | 1 | 3 | 95 | 55.59 ± 2.00 | 0.697 ± 0.063 | −43.3 ± 0.656 |
| 2-6 | 2 | 1 | 1 | 96 | 380.4 ± 29.14 | 0.854 ± 0.083 | −37.8 ± 1.00 |
| 2-7 | 3 | 1 | 1 | 95 | 380.6 ± 22.92 | 0.842 ± 0.034 | −23.1 ± 0.764 |
| 2-8 | 1 | 2 | 2 | 95 | 89.83 ± 1.689 | 0.384 ± 0.018 | −40.2 ± 1.6 |

From Table 3, when using chain-structured linalool, considering the comprehensive results of particle size, polymer dispersion index, and zeta potential, the nanocarrier composition of Group 2-8 was selected for subsequent experiments.

Next, Groups 1-8 and 2-8 were loaded with different proportions of cannabidiol, and the particle size, polymer dispersion index (PDI), zeta potential (Zeta Potential), encapsulation efficiency (EE), and drug loading (DL) of the nanocarrier compositions were compared. In addition, lecithin liposomes prepared using only lecithin and a surfactant and loaded with cannabidiol were used as a control group for comparison. The experimental results are shown in Tables 4 and 5.

TABLE 4

| | | | | | | | | Zeta |
|---|---|---|---|---|---|---|---|---|
| | Lecithin | Terpenes | Tween 80 | CBD | water | Particle size | | potential |
| Group | (wt %) | (wt %) | (wt %) | (wt %) | (wt %) | (nm) | PDI | (mV) |
| control | 2 | 0 | 2 | 0.3 | 95.7 | 243 ± 3.36 | 0.291 ± 0.021 | −29.5 ± 0.40 |
| group | | | | | | | | |
| 1-9 | 1 | 3 | 3 | 0.5 | 92.5 | 145.3 ± 1.31 | 0.276 ± 0.01 | −32.9 ± 0.38 |
| 1-10 | 1 | 3 | 3 | 1 | 92 | 153.4 ± 1.85 | 0.386 ± 0.01 | −24.3 ± 1.46 |
| 1-11 | 1 | 3 | 3 | 2 | 91 | 272 ± 10.10 | 0.495 ± 0.09 | −41.3 ± 1.00 |
| 2-9 | 1 | 2 | 2 | 0.5 | 94.5 | 148.7 ± 1.97 | 0.242 ± 0.01 | −22 ± 0.61 |
| 2-10 | 1 | 2 | 2 | 1 | 94 | 213.5 ± 0.21 | 0.331 ± 0.03 | −31.1 ± 2.15 |
| 2-11 | 1 | 2 | 2 | 2 | 93 | 245.8 ± 1.11 | 0.264 ± 0.03 | −49.4 ± 0.87 |

TABLE 5

| | | | | | | Encapsulation | |
|---|---|---|---|---|---|---|---|
| | Lecithin | Terpenes | Tween 80 | CBD | water | efficiency | Drug loading |
| Group | (wt %) | (wt %) | (wt %) | (wt %) | (wt %) | (EE)(%) | (DL)(%) |
| control | 2 | 0 | 2 | 0.3 | 95.7 | 59.61 ± 2.66 | 4.41 ± 0.22 |
| group | | | | | | | |
| 1-9 | 1 | 3 | 3 | 0.5 | 92.5 | 93.82 ± 1.05 | 9.48 ± 0.11 |
| 1-10 | 1 | 3 | 3 | 1 | 92 | 60.39 ± 1.25 | 14.36 ± 0.32 |
| 1-11 | 1 | 3 | 3 | 2 | 91 | 41.53 ± 1.73 | 18.68 ± 0.98 |
| 2-9 | 1 | 2 | 2 | 0.5 | 94.5 | 98.01 ± 0.48 | 11.7 ± 0.03 |
| 2-10 | 1 | 2 | 2 | 1 | 94 | 95.85 ± 1.2 | 20 ± 0.23 |
| 2-11 | 1 | 2 | 2 | 2 | 93 | 88.42 ± 2.26 | 26.89 ± 0.68 |

The results in Tables 4 and 5 show that the inclusion of different types and ratios of terpene compounds in the nanocarrier compositions not only affects the microstructure of the nanocarrier compositions, but also further influences the encapsulation efficiency (EE) and drug loading (DL) of the nanocarriers. Table 5 also reveals that the lecithin liposomes prepared using only lecithin and a surfactant (control group) exhibit significantly lower EE and DL than the nanocarrier compositions containing terpene compounds (1-9 to 1-11, 2-9 to 2-11), indicating that the addition of terpene compounds can effectively improve both EE and DL.

Furthermore, in nanocarrier compositions in Groups 1-9 to 1-11, which incorporated the cyclic structure of D-limonene, it was found that even with increasing dosage of cannabidiol, the encapsulation efficiency was not improved, and even precipitation occurred (in Groups 1-10 and 1-11). Furthermore, the particle size and polymer dispersion index (PDI) of the nanocarrier compositions also increased. Based on a comprehensive consideration of high encapsulation efficiency, small particle size, and PDI values at an appropriate dosage, Group 1-9 were selected as the optimal group.

Furthermore, among the nanocarrier compositions in Groups 2-9 to 2-11 containing chain-structured linalool, it was found that although the particle size and PDI value of the nanocarrier composition 2-11 were larger, its encapsulation efficiency was still over 80%, and its drug loading capacity was also higher than that of the nanocarrier compositions in Groups 2-9 and 2-10, and no visible precipitation was observed. Therefore, after comprehensive consideration, the nanocarrier composition of Group 2-11 was selected as the optimal composition.

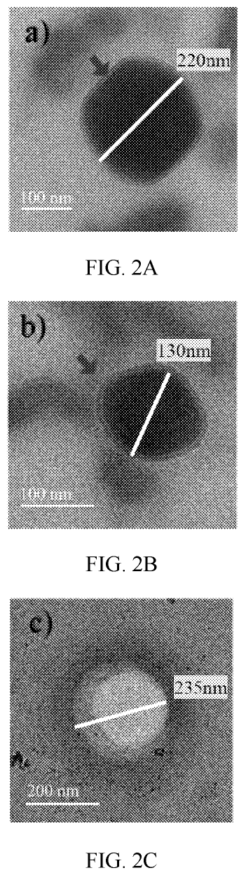

Please refer to FIG. 2A to FIG. 2C, which shows the structures of the nanocarrier composition containing cyclic terpene compounds and linear terpene compounds of the present disclosure, as well as lecithin liposomes without terpene compounds, observed under an electron microscope under negative staining. FIG. 2A shows the lecithin liposomes without terpene compounds, FIG. 2B shows the nanocarrier composition containing D-limonene, and FIG. 2C shows the nanocarrier composition containing linalool.

FIGS. 2A, 2B, and 2C, respectively, show the results of negative staining the nanocarriers from the control group, Groups 1-9, and 2-11 with phosphotungstic acid (PTA). After negative staining, the hydrophilic regions are more easily visible as the negatively stained black areas, while the lipophilic regions appear as a lighter dark gray. Therefore, it is clear that the structures in FIGS. 2A and 2B are liposomes. Since the hydrophobic ends of the micelles face inward and the hydrophilic ends face outward, the interior is less susceptible to negative staining, appearing as light gray areas in FIG. 2C, while the hydrophilic ends on the outside are negatively stained and appear darker gray. Therefore, FIG. 2A to FIG. 2C further show that when loaded with cannabidiol, the structures of each nanocarrier retain the intact morphology of a liposome or micelle.

Furthermore, as shown in FIG. 2A to FIG. 2C, the liposomes treated with terpenes (FIG. 2B) exhibited significantly increased outer ring thickness and a more regular morphology compared to the control group (FIG. 2A) without terpenes. Such morphological enhancement is consistent with improved encapsulation efficiency and drug loading capacity, as well as extended storage stability observed in subsequent experiments, indicating that terpenes enhance the structural stability of liposomes by altering the arrangement of the lipid bilayer.

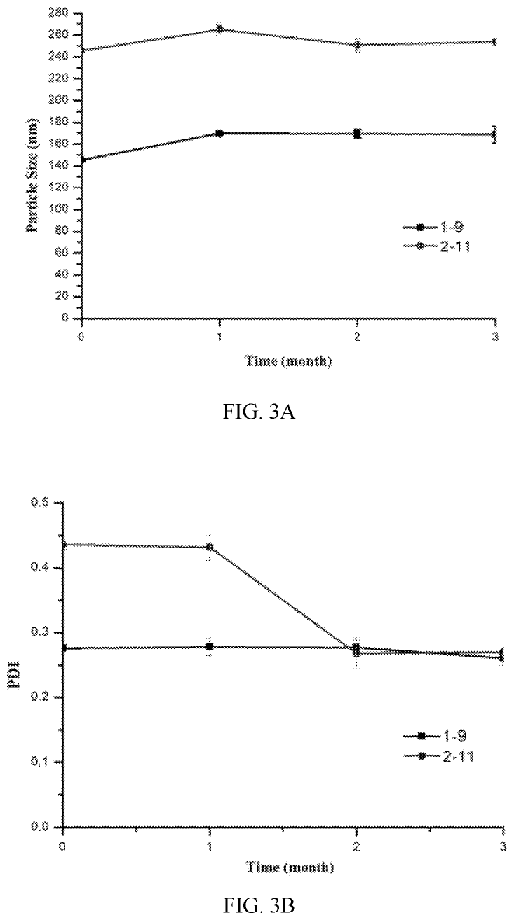

Please also refer to FIG. 3A to FIG. 3B, which are schematic diagrams illustrating the analysis of the particle size and polymer dispersion index of the nanocarrier composition containing cyclic terpene compounds and linear terpene compounds of the present disclosure over a period of three months. FIG. 3A is an analysis diagram of the particle size change, and FIG. 3B is an analysis diagram of the polymer dispersion index change.

FIG. 3A shows that the particle size of the nanocarrier composition of Group 1-9 containing D-limonene and the nanocarrier composition of Group 2-11 containing linalool remained unchanged after three months. FIG. 3B also shows that the PDI values the nanocarrier composition of Group 1-9 containing D-limonene remained almost unchanged over the three months, indicating that the polymer in the nanocarriers was stable, with essentially no change in molecular weight distribution. The PDI values of the nanocarrier composition of groups 2-11 containing linalool continued to decrease over the three months, indicating that the molecular weight distribution of the polymer in the nanocarriers gradually became more uniform over time and stabilized after two months, showing no significant change.

Therefore, as can be seen from FIG. 3A and FIG. 3B, the nanocarrier composition containing terpene compounds, whether in the form of liposomes or micelles, can remain stable for a long period of time, which is more conducive to long-term storage of drugs and maintenance of drug activity when loaded.

### Example 3

As shown in the experimental results of Example 2, the nanocarrier composition containing a specific type and ratio of terpene compounds not only has a good encapsulation efficiency and drug loading capacity, but also maintains the particle size and PDI value of the nanocarrier itself within an acceptable range. Next, to confirm whether the nanocarrier composition containing terpene compounds has similar encapsulation capabilities for other types of drugs in addition to cannabidiol, loading tests were conducted on 11 different types of compounds, including cannabinoids, flavonoids, carotenoids, triterpenes, polyphenols, alkaloids, fatty acids, fat-soluble vitamins, polysaccharides, volatile oils, and amino acids, totaling 32 compounds. Each test was repeated three times and the average value was calculated. This confirmed that the nanocarrier composition containing terpene compounds has excellent encapsulation capabilities for various compounds. The results are shown in Table 6.

TABLE 6

| | | control group | | 1-9 | | 2-11 | |
|---|---|---|---|---|---|---|---|
| | | Particle size | | Particle size | | Particle size | |
| | Compound Type | (nm) | PDI | (nm) | PDI | (nm) | PDI |
| 0 | Unloaded drug | 256 | 0.517 | 109.4 | 0.388 | 184.6 | 0.424 |
| | nanocarriers | | | | | | |
| | Cannabinoids | | | | | | |
| 1 | cannabidiol | 243 | 0.291 | 145.3 | 0.276 | 245.8 | 0.264 |
| | Flavonoids and Flavonols | | | | | | |
| 2 | Astilbin | 262.1 | 0.514 | 117.6 | 0.399 | 179.8 | 0.489 |
| 3 | Rutin | 246.4 | 0.558 | 116.4 | 0.442 | 147.6 | 0.374 |
| 4 | Quercetin | 246.2 | 0.666 | 116.7 | 0.385 | 171.8 | 0.405 |
| 5 | Dihydromyricetin | 250.9 | 0.541 | 122.6 | 0.39 | 147.2 | 0.487 |
| 6 | Silymarin | 254.4 | 0.627 | 118.2 | 0.408 | 128.7 | 0.411 |
| | Carotenoids | | | | | | |
| 7 | Lutein | 271.2 | 0.493 | 140.2 | 0.469 | 153.7 | 0.445 |
| 8 | Lycopene | 256.3 | 0.553 | 128.2 | 0.396 | 149 | 0.389 |
| 9 | Cryptoxanthin | 245.2 | 0.707 | 119.6 | 0.385 | 175 | 0.485 |
| 10 | Zeaxanthin | 251.5 | 0.54 | 121.7 | 0.452 | 168.1 | 0.45 |
| 11 | Capsanthin oil | 265.5 | 0.579 | 148.4 | 0.294 | 161.5 | 0.328 |
| | Triterpenes and Terpenoids | | | | | | |
| 12 | Centella asiatica | 251 | 0.603 | 121 | 0.404 | 171.2 | 0.396 |
| 13 | Boswellic acid | 257.1 | 0.613 | 123.5 | 0.421 | 162.3 | 0.401 |
| 14 | Ganoderma lucidum | 249.1 | 0.48 | 125.4 | 0.422 | 152 | 0.368 |
| | triterpenes | | | | | | |
| 15 | Protopanaxadiol | 260.2 | 0.527 | 119.3 | 0.381 | 162.5 | 0.429 |
| | Polyphenolic Compounds | | | | | | |
| 16 | Curcumin | 250 | 0.567 | 121.9 | 0.412 | 154.6 | 0.425 |
| 17 | Tetrahydrocurcumin | 238.2 | 0.555 | 120.9 | 0.464 | 170 | 0.407 |
| 18 | Resveratrol | 248.1 | 0.642 | 124.8 | 0.408 | 148.3 | 0.446 |
| | Alkaloids and Derivatives | | | | | | |
| 19 | Pilocarpine hydrochloride | 224.7 | 0.49 | 121.5 | 0.354 | 165.4 | 0.359 |
| 20 | Dihydroavenate alkaloids | 251.6 | 0.495 | 121.7 | 0.402 | 148.2 | 0.431 |
| 21 | Indigo carmine | 252 | 0.58 | 124.3 | 0.436 | 158.5 | 0.422 |
| | Fatty Acids and Plant Oils | | | | | | |
| 22 | nervonic acid | 248.2 | 0.542 | 144.3 | 0.502 | 179.9 | 0.439 |
| 23 | Xanthoceras sorbifolia oil | 270.7 | 0.5 | 176.2 | 0.421 | 171.3 | 0.386 |
| 24 | Sea buckthorn fruit oil | 276.4 | 0.511 | 183.9 | 0.306 | 200.3 | 0.501 |
| 25 | Moringa seed oil | 268.8 | 0.491 | 181.9 | 0.334 | 219.5 | 0.537 |
| | Fat-Soluble Vitamins and Cofactors | | | | | | |
| 26 | Vitamin K2 | 251.6 | 0.511 | 121.8 | 0.424 | 155.1 | 0.378 |
| | Volatile Oils | | | | | | |
| 27 | cinnamon oil | 241.9 | 0.522 | 118.6 | 0.383 | 227.7 | 0.533 |
| 28 | mustard oil | 254.6 | 0.574 | 122.5 | 0.454 | 140.3 | 0.471 |
| 29 | Angelica Oil | 288.6 | 0.468 | 206.9 | 0.383 | 163.3 | 0.315 |
| | Amino Acids and Neurotransmitters | | | | | | |
| 30 | Gamma-aminobutyric acid | 239.5 | 0.63 | 122.3 | 0.364 | 172.5 | 0.345 |
| | (GABA) | | | | | | |
| | Polysaccharides | | | | | | |
| 31 | Ganoderma lucidum | 262.2 | 0.652 | 119.4 | 0.438 | 159.3 | 0.384 |
| | polysaccharide | | | | | | |
| | Quinone Cofactors | | | | | | |
| 32 | Pyrroloquinoline quinone | 246.5 | 0.528 | 160.6 | 0.554 | 158.3 | 0.382 |
| | (PQQ) | | | | | | |

The results in Table 6 show that nanocarrier compositions containing terpenes as regulators exhibit excellent encapsulation capabilities for 32 compounds of 11 different properties, including cannabinoids, flavonoids, carotenoids, triterpenes, polyphenols, alkaloids, fatty acids, fat-soluble vitamins, polysaccharides, volatile oils, and amino acids. After encapsulation, the nanocarrier compositions containing terpenes as regulators exhibited desirable particle sizes and PDI values, demonstrating the broad applicability across diverse active ingredients of nanocarrier compositions containing terpenes as regulators.

In summary, the nanocarrier composition with adjustable structure disclosed in the present disclosure, as a nanocarrier system, does have a significant innovative design and wide applicability, providing a novel and versatile solution in the field of drug delivery. By selecting terpene structures and adjusting their ratios, the nanocarrier morphology can be precisely tuned between liposomes and micelles, enabling dosage form optimization for either oral or transdermal delivery. This structural adjustability directly translates into improved therapeutic efficacy, extended storage stability, and broad applicability across diverse active pharmaceutical ingredients.

Through the description of the above-mentioned embodiments, the structure, operation module, method of use and possible technical effects of the present disclosure have been fully described. However, the above-mentioned embodiments are only preferred examples of the present disclosure and are not intended to limit the scope of the present disclosure. Those skilled in the art may make adjustments or modifications to the disclosed embodiments, material compositions or process methods without departing from the spirit and scope of the present disclosure, which still fall within the scope covered by the claims of the present disclosure. In addition, the description, examples and experimental data herein are mainly used to describe in detail the technical features and methods of the present disclosure, but according to the differences in specific application scenarios or needs, necessary optimizations and changes to the implementation methods may be applied by those skilled in the art without departing from the core concept of the present disclosure. It is to be understood that various modifications, equivalents, and alternatives may be made to the embodiments described herein without departing from the spirit and scope of the present disclosure, which is intended to be defined by the appended claims.

What is claimed is:

1. A nanocarrier composition with adjustable structure, comprising:
lecithin; a regulator; a surfactant; and water, wherein
the regulator is a terpene compound selected from a monocyclic terpene compound, a bicyclic terpene compound or a chain terpene compound; and
the surfactant is any one of anionic surfactant, cationic surfactant, zwitterionic surfactant, nonionic surfactant or ionic surfactant or a combination thereof.

2. The nanocarrier composition with adjustable structure of claim 1, wherein
the monocyclic terpene compound is selected from D-limonene, phellandrene, pulegone, sabinene, terpinene, DL-menthol, terpineol, humulene, and (−)-α-bisabolol;
the bicyclic terpene compound is selected from camphene, pinene, fenchone, (−)-borneol, camphor, fenchol, trans-caryophyllene, valencene, caryophyllene oxide, guaiol, cedrene, (+)-cedrol; and
the chain terpene compound is selected from linalool, nerol, β-myrcene, ocimene, nerolidol and farnesene.

3. The nanocarrier composition with adjustable structure of claim 1, wherein the surfactant is selected from any one of polysorbate, polyethylene glycol, polymethyl methacrylate, polyglycolide, polylactide, polycaprolactone, polylactide-polyethylene glycol, polycaprolactone-polyethylene glycol, vitamin E polyethylene glycol succinate, poloxamer, or polyoxyethylene hydrogenated castor oil, or a combination thereof.

4. The nanocarrier composition with adjustable structure of claim 1, wherein the terpene compound is a monocyclic terpene compound or a bicyclic terpene compound when the nanocarrier composition is to form a liposome structure.

5. The nanocarrier composition with adjustable structure of claim 1, wherein the terpene compound is a chain terpene compound when the nanocarrier composition is to form a micelle structure.

6. A method for preparing a nanocarrier composition with adjustable structure, comprising the following steps:
Step S1: dissolving lecithin, a regulator, and a surfactant in an organic solvent to form a mixed solution;
Step S2: evaporating and removing the organic solvent in the mixed solution to form a thin film containing the lecithin, the regulator, and the surfactant; and
Step S3: adding water to dissolve the thin film back, and apply energy to perform a hydration reaction to obtain the nanocarrier composition containing the regulator, wherein
the regulator is a terpene compound, and the terpene compound is selected from a monocyclic terpene compound, a bicyclic terpene compound or a chain terpene compound;
the organic solvent is selected from any one of methanol, n-hexane, ethanol, isopropanol, acetone, formic acid, acetic acid, acetonitrile, dichloromethane, dimethylformamide or tetrahydrofuran, or a combination thereof;
the surfactant is any one of anionic surfactant, cationic surfactant, zwitterionic surfactant, nonionic surfactant or ionic surfactant or a combination thereof; and
the method for applying energy includes stirring, vortexing or ultrasonication in a water bath.

7. A method for loading a drug using a nanocarrier composition with adjustable structure, wherein
the drug is selected from the group consisting of cannabinoids, flavonoids, carotenoids, triterpenoids, polyphenols, alkaloids, fatty acids, fat-soluble vitamins, polysaccharides, volatile oils, and amino acid compounds; and
the nanocarrier composition with adjustable structure comprising lecithin, a regulator, a surfactant and water, wherein
the regulator is a terpene compound, and the terpene compound is selected from a monocyclic terpene compound, a bicyclic terpene compound or a chain terpene compound; and
the surfactant is any one of anionic surfactant, cationic surfactant, zwitterionic surfactant, nonionic surfactant or ionic surfactant or a combination thereof.
